(19) United States
(12) Patent Application Publication
(10) Pub. No.: US 2026/0215748 A1
(43) Pub. Date: Jul. 30, 2026

(54) SYSTEM, METHOD AND COMPUTER-ACCESSIBLE MEDIUM FOR EFFECTUATING REAL TIME IMAGE-BASED PHYSIOLOGY AND/OR CORONARY MEASUREMENTS

(71) Applicant: KONINKLIJKE PHILIPS N.V., EINDHOVEN (NL)

(72) Inventors: Vivek WALIMBE, Westford, MA (US); Eman NAMATI, Concord, MA (US); Damon DEPAOLI, Arlington, MA (US)

(21) Appl. No.: 19/147,516

(22) PCT Filed: Jan. 12, 2024
(86) PCT No.: PCT/US2024/011364

§ 371 Date: Jul. 11, 2025

Related U.S. Application Data
(60) Provisional application No. 63/438,658, filed on Jan. 12, 2023.
(60) Provisional application No. 63/546,327, filed on Oct. 30, 2023.

(51) Int. Cl. A61B 6/50; A61B 6/00; A61B 6/58

(52) U.S. Cl. CPC A61B 6/504; A61B 6/481; A61B 6/5211; A61B 6/582

(57) ABSTRACT

Exemplary systems, apparatus and methods, and computer-accessible medium can be provided for effectuating one or more image-based physiology measurements. For example, it is possible to position a reference object within an imaging field of view of an X-ray radiation source, and in a vicinity of at least one section of a body or a target object. It is further possible to provide X-ray radiation to such portion of the body or the target object, as well as to the reference object(s). Further, it is possible to generate at least one X-ray image of the portion(s) that contains an image of the reference object based on (i) a first radiation provided from the portion, and (ii) a second radiation provided from the reference object.

### CROSS REFERENCE TO RELATED APPLICATION(S)

This application relates to and claims the benefit of priority from U.S. Provisional Patent Application No. 63/438,658, filed on Jan. 12, 2023, and U.S. Provisional Patent Application No. 63/546,327, filed on Oct. 30, 2023, the entire disclosures of which are incorporated herein by reference in their entireties.

### FIELD OF THE DISCLOSURE

The present disclosure relates generally to the imaging technology, and more particularly, to systems, methods, and computer-accessible medium for effectuating one or more image-based physiology and/or coronary measurements.

### BACKGROUND INFORMATION

To perform an image-based assessment of coronary artery structure (e.g., Quantitative coronary angiography) and physiology (e.g., angiography-derived coronary physiology), a first requirement can be to obtain and use fluoroscopy/angiography image stacks. Most often multiple image stacks are used that have been taken at two known angles. A second requirement can be to obtain and use traditional metadata, such as source-to-sample distance, system-specific magnification factors and the resulting image-pixel size to adjust measurements based on the imaging conditions and imaging system characteristics.

Most prior procedures can derive X-ray geometric information for the second requirement from the metadata associated with one or more angiography image Digital Imaging and Communications in Medicine (DICOM) files (e.g., at each imaging angle). A DICOM file from the health information management system is not typically obtained in real-time, which adds additional user interaction and time, and limits the advantage of a non-invasive angiography-derived coronary assessment (ADCA) methods (e.g., Angiography-derived geometry and/or structural measurements (e.g., quantitative coronary angiography (QCA)), absolute or relative coronary flow (CF), fractional flow reserve (FFR), Instantaneous wave free ratio/resting full cycle ratio (iFR/RFR), Index of microcirculatory resistance (IMR), Hyperemic microvascular resistance (HMR), Hyperemic stenosis resistance (HSR), coronary flow reserve (CFR). Furthermore, while angiography-derived physiology (ADP) has shown promising accuracy in comparison to ground-truth physiology measurements in ideal conditions, inconsistent image quality, system-dependent factors, and additional user interactions and procedure time have previously reduced its performance and applicability in the operating room (OR).

Preferably, X-ray geometric metadata would be available to the processing system directly from the fluoroscope to facilitate a rapid image-based ADCA. Unfortunately, currently available technologies only provide an open live-stream of angiography image data optimized for display on an OR monitor (e.g., via a video-out port). Furthermore, this live-stream of image data does not typically transmit the necessary geometric metadata to perform ADCA.

Accordingly, there is a need to provide apparatuses, methods and/or computer-accessible medium to perform real-time ADCAs as well as generate and transmit the necessary geometric metadata about the imaging and system parameters.

### SUMMARY OF EXEMPLARY EMBODIMENTS

Such issues and/or deficiencies can at least be partially addressed and/or overcome by providing system, method and computer-accessible medium for effectuating one or more ADCAs, in accordance with various exemplary embodiments according to the present disclosure.

According to an exemplary embodiment of the present disclosure, an X-ray imaging system can be provided along with a processing system. Such exemplary processing system can be configured to perform ADCA, such as e.g., QCA and/or ADP, using one or more images from a live-stream of angiography images from the X-ray system. In this exemplary embodiment, ADP can be performed in real-time using the video-out signal of an X-ray imaging system directly.

In an exemplary embodiment, the X-ray imaging system can comprise, e.g., a X-ray radiation source configured to generate an X-ray radiation, at least one reference object (which can be, e.g., partially or substantially-fully radiopaque) within an imaging field-of-view of the X-ray radiation source and configured to receive the X-ray radiation, and at least one computer processor (e.g., an X-ray image formation device) configured to generate an X-ray image containing an image of the reference object based on a first radiation received and then attenuated by at least one portion of a body and a second radiation received and then attenuated by the reference object, and then such attenuated radiation is provided thereby. For example, a contrast agent (which can be, e.g., a radiopaque contrast agent) can also be provided into the body prior to the body receiving the X-ray radiation.

In an exemplary embodiment of the present disclosure, the radiopaque contrast agent can be a partially radiopaque contrast agent. The exemplary imaging system can further comprise an X-ray detector in optical and/or data communication with the computer processor(s). The radiopaque reference object can be a partially radiopaque reference object. The radiopaque reference object can be or include a patterned object of known and/or predetermined dimensions. The radiopaque reference object can be or include a three-dimensional object, and/or can be a triangle-shaped object, a circular-shaped object, a square-shaped object, a grid-shaped object, and/or a polygonal-shaped object. The radiopaque reference object can be an intravascular instrument (e.g., a catheter) and/or a portion thereof.

In another exemplary embodiment of the present disclosure, the radiopaque reference object can be arranged on an operation platform on which the body (e.g., a patient) is positioned. The radiopaque reference object can be a portion of (e.g., placed on) an imaging catheter, e.g., on a surface thereof. The radiopaque reference object can also (or alternatively) be placed on a front portion and/or a back portion of the body of the target object (e.g. stuck to the body). The radiopaque reference object can comprise a visible identifying tag (e.g., QR code, which can be readable using an optical QR reader). Similarly, the radiopaque reference object can comprise a radiopaque identifying tag (e.g., a radiopaque QR code which can be readable using the X-ray image of the reference object). The radiopaque reference object can be composed of, at least partially, metal and/or plastic. In still another exemplary embodiment of the present disclosure, the imaging system can further comprise a patient identification (ID) tag. Imaging and system metadata (e.g., the pixel sizes of the X-ray image) can be obtained from the image of the radiopaque reference object (e.g., using an automated image analysis).

According to another exemplary embodiment of the present disclosure, a method of performing ADP measurements within a body can be provided. The exemplary method can comprise, e.g., disposing a reference object (which can be at least partially radiopaque) within an imaging field-of-view of an X-light generator and in vicinity of at least one portion of a body or a target object, imaging at least one portion of a body provided within the imaging field-of-view, providing an X-ray radiation to such portion and the reference object, and generating at least one X-ray image of the imaging field-of-view, and such portion containing an image of the reference object based on (i) a first radiation received and attenuated by this portion, and (ii) a second radiation provided from the reference object, and then such attenuated radiation is provided thereby.

According to another exemplary embodiment, it is possible to measure a physiological feature of such section. The reference object (which can be partially or fully radiopaque) can be shaped or designed to provide information to facilitate the measurement. In another exemplary embodiment of the method according to the present disclosure, a contrast agent (which can be at least partially radiopaque) within a lumen of a body,

According to still another exemplary embodiment of the present disclosure, a method for tracking a catheter within a body can be provided. The exemplary method can comprise disposing a reference object (which can be at least partially radiopaque) within an imaging field-of-view of an X-light generator and in vicinity of at least one portion of a body or a target object.

The partially radiopaque reference object can comprise an interpretable symbol or combination of symbols that facilitate an image-based detection and tracking of or within at least one portion of the body or the target object based on a radiation received and attenuated by at least one portion the body or the target object in response of X-ray radiation impacting such portion, and then such attenuated radiation is provided thereby.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a diagram of an exemplary prior art system for effectuating ADP measurements;

FIG. 2 is a diagram of an exemplary system for effectuating ADP measurements according to an exemplary embodiment of the present disclosure;

FIG. 3 is a set of representations of exemplary angiogram diagrams with and without a reference object according to according to an exemplary embodiment of the present disclosure;

FIG. 4 is a set of representations of exemplary diagrams of reference objects that can be used by the exemplary apparatus/system/method/device/computer-accessible medium in a visible field of view according to another exemplary embodiment of the present disclosure;

FIG. 5 is a set of representations of exemplary diagrams of multiple reference objects that can be used by the exemplary apparatus/system/method/device/computer-accessible medium in a visible field of view according to another exemplary embodiment of the present disclosure;

FIG. 6 is a set of representations of exemplary diagrams of 2 and/or 3 dimensional (3D) reference objects that can be used by the exemplary apparatus/system/method/device/computer-accessible medium in a visible field of view according to another exemplary embodiment of the present disclosure;

FIG. 7 is a diagram showing an exemplary apparatus/system/computer-accessible medium having a reference object for imaging at least one portion of a patient according to another exemplary embodiment of the present disclosure;

FIG. 8 is a diagram of an exemplary angiogram showing a catheter and a progression thereof within a lumen of a body that can be implemented in the apparatus/system/computer-accessible medium according to yet another exemplary embodiment of the present disclosure;

FIG. 9 is a diagram of an exemplary angiogram showing markers that can be implemented with a catheter utilized in and/or with the apparatus/system/computer-accessible medium according to yet another exemplary embodiment of the present disclosure;

FIG. 10 is a set illustration of exemplary angiograms providing exemplary flow measurement results with and without a reference object, respectively, that is implemented in the system/apparatus/device/method/computer-accessible medium according to an exemplary embodiment of the present disclosure;

FIG. 11 is a cross-sectional view of an illustration providing further flow measurement results with and without a reference object that is implemented in the system/apparatus/device/method/computer-accessible medium according to an exemplary embodiment of the present disclosure;

FIG. 12 is a set of diagrams providing exemplary flow measurements with different placement angles of a reference object that is implemented in the system/apparatus/device/method/computer-accessible medium according to an exemplary embodiment of the present disclosure;

FIG. 13 is a set of diagrams illustrating reference objects in the morphology and ADP representations, respectively, used for multiple imaging modes that are implemented in the system/apparatus/device/method/computer-accessible medium according to an exemplary embodiment of the present disclosure;

FIG. 14 is a set of diagrams illustrating reference objects in the morphology and ADP representations, respectively, used for multiple imaging modes that are implemented in the system/apparatus/device/method/computer-accessible medium according to another exemplary embodiment of the present disclosure;

FIG. 15 is a flow diagram of a process for computing a fractional flow reserve (FFR) based on the display image as exists in the prior art;

FIG. 16 is a flow diagram of a process for computing the fractional flow reserve (FFR) based on the display image from the video out port on an intravascular imaging system that are implemented in the system/apparatus/device/method/computer-accessible medium according to another exemplary embodiment of the present disclosure;

FIG. 17A is a flow diagram of a process for angiography-derived measurements based on contrast agent filling lumen of a vessel according to an exemplary embodiment of the present disclosure;

FIG. 17B is a flow diagram of a process that utilizes at least one image sequence to measure flow and pressure, optionally incorporating side branches which can also cause a pressure drop that can improve the accuracy of the FFR measurement according to an exemplary embodiment of the present disclosure; and

FIG. 18 is a flow diagram of a process for angiography-derived measurements based on an angiography image, a user input, and an automatically detected reference object according to an exemplary embodiment of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the certain exemplary embodiments illustrated in the figures and the appended claims.

### DETAILED DESCRIPTION OF EXAMPLE EMBODIMENTS

The following description of embodiments provides non-limiting representative examples referencing numerals to particularly describe features and teachings of different aspects of the present disclosure. The embodiments described should be recognized as capable of implementation separately, or in combination, with other exemplary embodiments from the description of the exemplary embodiments. A person of ordinary skill in the art reviewing the description of the exemplary embodiments should be able to learn and understand the different described aspects of the present disclosure. The description of the exemplary embodiments should facilitate understanding of the disclosure to such an extent that other implementations, not specifically covered but within the knowledge of a person of skill in the art having read the description of embodiments, would be understood to be consistent with an application of the present disclosure.

For example, according to an exemplary embodiment of the present disclosure, system, apparatus, method, device and/or computer-accessible medium can be provided that can acquire the metadata in real-time by, for example, providing (e.g., burning) a metadata text into the output angiography image stream from the X-rays system, and deploying optical character recognition (OCR) methods on the ADCA system to recognize, read and/or analyze the image-text.

According to another exemplary embodiment of the present disclosure, system, apparatus, method, device and/or computer-accessible medium can be provided that can acquire the metadata in real-time by, for example, placing a well-known radiopaque referencing tool in the X-ray field of view (i.e., without interfering with the patient's arteries of interest). Such a reference tool can be designed to enable full reconstruction of the X-ray system geometry which can be important to accurately determine or calculate the associated ADP in a system agnostic manner.

According to some exemplary embodiments of the present disclosure, the system, apparatus, method, device and/or computer-accessible medium described herein can facilitate rapid/live ADCA without the need for transferring angiography system metadata (e.g., without a DICOM, and/or without the use of a network connection); higher accuracy imaging measurements tied directly to patient position (opacity, imaging angle, pixel size, sample distance, etc.); fluoroscopy/angiography-system-independent algorithm calibration (e.g., allowing competitive technological innovation); reducing dependence on system manufacturers and allowing system independent image normalization; single-slice angiography-based FFR at non-confined angles (e.g. at angles that are uncommon, at angles that capture the minimum lumen area (MLA) optimally) using the measured angle of the reference tool; providing tracking for “single-use” payment by using unique serialization embedded in the referencing tool and decipherable by the ADCA system; ability to sell a single-use referencing tool tied to the procedure (i.e., for payments); and reducing dependance on image-text (undesired) and additional DICOM interconnection.

In an exemplary embodiment of the present disclosure, the referencing object/tool can be composed of, at least in part, metal, polymer, plastic and/or any material with a controlled/tuned opacity for visibility on an x-ray. The referencing object/tool can have letters or numbers or any pattern that allows an image processing algorithm to read identifiable information from the object. The referencing object/tool can be multiple disconnected objects. The referencing object/tool can be an object that is installed in the OR (e.g., on an operating table).

In another exemplary embodiment of the present disclosure, the referencing object/tool can be or include a disposable object (i.e., a QR scannable item) for tracking payments. The referencing object/tool can be configured and/or structured to stick to patient or table or be weighted as to not move. The referencing object/tool can also be provided on a guide catheter (i.e., a specific guide catheter designed for such a purpose) or any other intravascular object. The referencing object/tool can further provide information to infer pixel size or angle of the imaging conditions (e.g., the source/detector angle relative to an operating table or patient), and/or to infer a distance from the source to the sample and/or the source to the detector.

In yet another exemplary embodiment of the present disclosure, the referencing object/tool can provide information regarding at least one portion of the patient, the model of the X-ray system, the type of ADCA (e.g., Angiography-derived: structural measurements, absolute or relative coronary flow (CF), fractional flow reserve (FFR), Instantaneous wave free ratio/resting full cycle ratio (iFR/RFR), Index of microcirculatory resistance (IMR), Hyperemic microvascular resistance (HMR), Hyperemic stenosis resistance (HSR), coronary flow reserve (CFR) and/or a single plane/angle ADCA (e.g., single angle angiography-derived FFR). The exemplary referencing object/tool can also provide information to improve optical coherence tomography (OCT) measurements (e.g., Stent/side branch measurements), and/or facilitate real-time angiography and/or physiology measurements.

In still another exemplary embodiment of the present disclosure, the referencing object/tool can facilitate and/or work together with the computer-accessible medium which includes a software module to utilize a different program/mode if detected. The referencing object/tool can utilize and/or implement a different analysis method (e.g., for angiography-derived FFR) and/or payment scheme, if detected. Additionally, the referencing object/tool can have a grid pattern, a square pattern, a triangular pattern and/or any geometric shape that can facilitate an inference of any of the previous points. The referencing object/tool can have two-dimensional features, three dimensional features, and/or other features that are differentiated by radiopacity in addition to structure.

As described above, unlike DICOM which is standard in the medical image format having information on the pixel size and all other information that is needed to calculate the different flow measurements, the exemplary system, apparatus, method, device and/or the computer-accessible medium described herein, can comprise and/or utilize a reference object with a controllable opacity into the imaging field of view. That reference object can be a single-use item. For example, when a clinician provides a reference object onto a patient like a sticker, the size of that reference object when imaged using an X-ray system will contain information on the size of one or more of pixels (and/or even for each of the pixels). The reference object when imaged using an X-ray system can also provide information about the distance from the sample to the source, the angle based on the orientation, and the image. Computer vision can be used to detect such information automatically, and the reference object can have a radiopaque identifying tag (e.g., a barcode) in it. The reference object can have a design indicating a size from which each pixel size can be inferred, such as how many microns each pixel equals. The reference object can be configured to provide a way to perform a payment scheme where it can be a single scan. The reference object can be provided as a sticker on the patient, which can provide a way to keep track of use of a certain software.

In one exemplary embodiment, the reference object can be placed between an emitter and a detector in the imaging field of view, e.g., at a position of above or below the patient so that the size of the reference object and the distance of the reference object from the emitter/detector can be known, from which each image pixel size can be obtained. For example, in angiography-derived FFR flow measurements, the reference object can be at least partially or fully radiopaque markers (e.g., which can resemble a small ball), by referencing to which the image size can be obtained. The reference object can be configured to have a QR code, which can be placed on a patient and then peeled off and scanned (e.g., optically or physically), thereby payment being tracked. Such exemplary information can be transmitted directly to a local computer system and does not have to be sent to a cloud platform from which that information is later retrieved for further processing—this exemplary scheme can reduce time consumption for processing such information. The reference object can be arranged as a grid on a platform (e.g., a table) on which a patient is provided. The reference object can also be a sticker that can be placed on any portion (e.g., chest/back/neck) of the patient or on a gown that the patient is wearing, as if the reference object is in the field of imaging view.

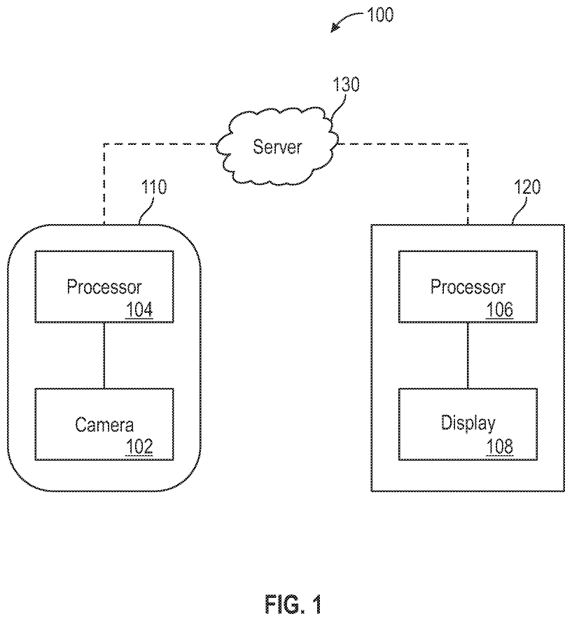

FIG. 1 shows a diagram of an exemplary prior art system 100 for ADCA measurements. As shown in the prior art system 100, an imaging system 110 (e.g., an X-ray system) can be provided that can include a camera 102 and a processor 104 controlling the camera 102 for imaging a patient. The imaging data/information of the imaging system 110 can be transmitted to a remote or cloud platform such as a cloud server 130. The system 100 can further include an image processing system 120 that can include a display 108 for displaying images/data and a processor 106 controlling the display 108. The image processing system 120 can retrieve the imaging data/information of the imaging system 110 from the server 130. Such exemplary prior art system 100 is not a real time image/data processing system, and can be time consuming for processing images/data due to the data/image transmissions between the imaging system 110, the cloud server 130 and the image processing system 120.

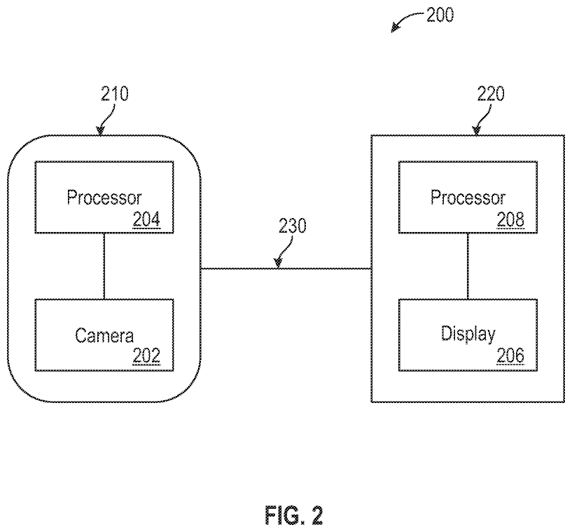

FIG. 2 shows a diagram of an exemplary system 200 for ADCA measurements according to an exemplary embodiment of the present disclosure. The exemplary system 200 can comprise an imaging subsystem 210 and an image processing subsystem 220. As shown in in FIG. 2, the imaging system 210 (which can be or include, e.g., an X-ray system) can comprise a camera (e.g. an X-ray source and detector) 202 and a processor 204 which can be configured to control and/or interact with the camera 202 for imaging an patient and transmitting the resulting image (e.g., to a server or a display). The imaging data/information of the imaging system 210 can be transmitted via a transmission medium 230 (e.g., a data cable or a wireless network) from an output on the imaging system (e.g., a video-out port) to the image processing system 220 that can comprise, e.g., a display 206 which can display images/data and at least one processor 208 which can be configured to control the display 206. The image processing system may be configured to perform ADCA using the resulting images transmitted by the transmission medium. In contrast to the exemplary prior art system 100 shown in FIG. 1, the exemplary system 200 can avoid transmitting the imaging data/information of the imaging system 210 to a remote or platform (e.g., a local server). Instead, the imaging data/information of the imaging system 210 can be transmitted-directly and real time-via the transmission medium 230 (e.g., a data cable or a wireless network) to the image processing system 220, which can improve the efficiency of image processing. In some embodiments, the image processing system may be an intravascular imaging system (e.g., An IVOCT, IVUS, NIRS, Fluorescence, Reflectance, Raman imaging system)

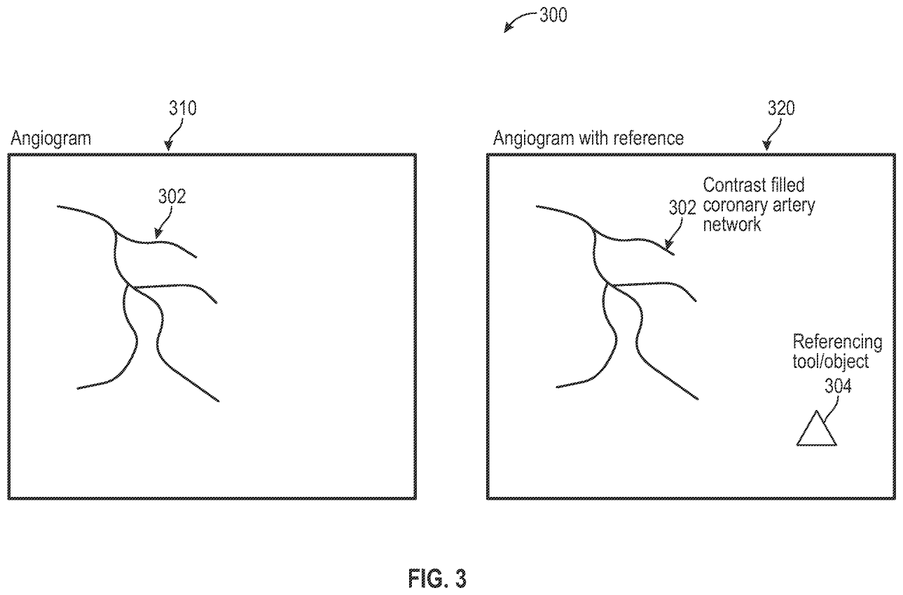

FIG. 3 shows a set of exemplary angiogram diagrams 300 of at least one portion of a body, with and without a reference object, respectively according to according to an exemplary embodiment of the present disclosure. A diagram 310 is an illustration of an angiogram in which a coronary artery network 302 is provided with a contrast agent and provided without a reference object, which can be a conventional angiogram. A diagram 320 is an illustration of an angiogram diagram of a contrast-filled coronary artery network 302 which also includes and utilizes a reference object 304, that can be implemented in the system, apparatus, method, device and/or computer-accessible medium according to the exemplary embodiments of the present disclosure, as described herein. The reference object 304 can be any shape and/or size object, e.g., and as shown in the example of FIG. 3 having a triangle shape.

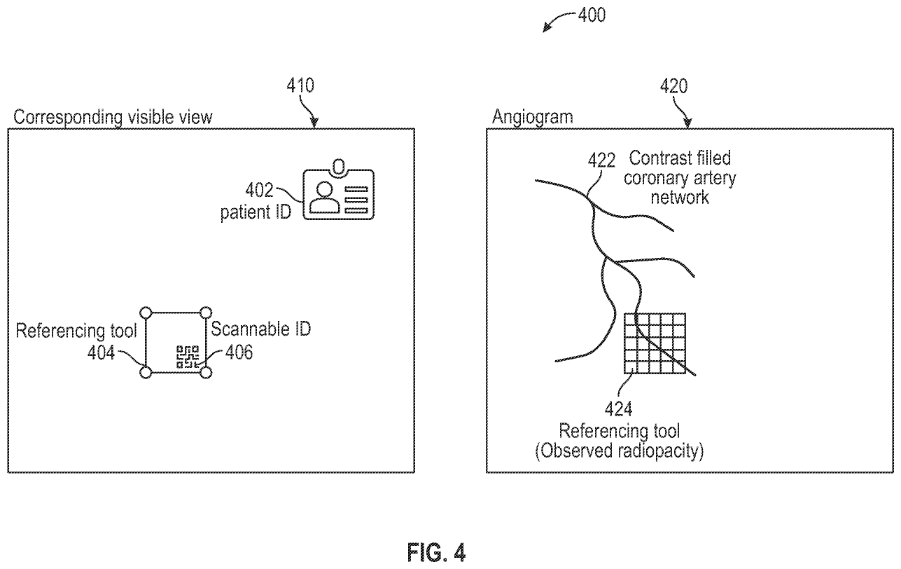

FIG. 4 shows a set of representations of exemplary diagrams 400 of reference objects that can be used by the exemplary apparatus/system/method/device/computer-accessible medium in a visible field of view according to another exemplary embodiment of the present disclosure. A diagram 410 is an illustration of an angiogram that can comprise a patient ID 402 in addition to a reference object/tool 404. The patient ID 402 can contain or be associated with information on a patient such as name, age, and so forth. The reference object 404 can further comprise a optically-scannable ID 406, such as, e.g., a QR code which can be associated with information regarding the imaging system and/or the patient and/or the procedure. A diagram 420 is an illustration of an angiogram that shows a reference object 424 and a contrast-filled coronary artery network 422. A reference object 424 shown in diagram 420 can be arranged as a grid. In this example, the diagram 410 shows the patient ID 402 and the referencing tool/object 404 without the blood vessel (e.g., artery) seen. When X-ray radiation is provided on the patient, the diagram 420 shows the blood vessel filled with contrast (e.g., the contrast filled coronary artery network 422) and the reference object 424 (because it is radiopaque), and without the patient ID tag 402 (because it is not radiopaque).

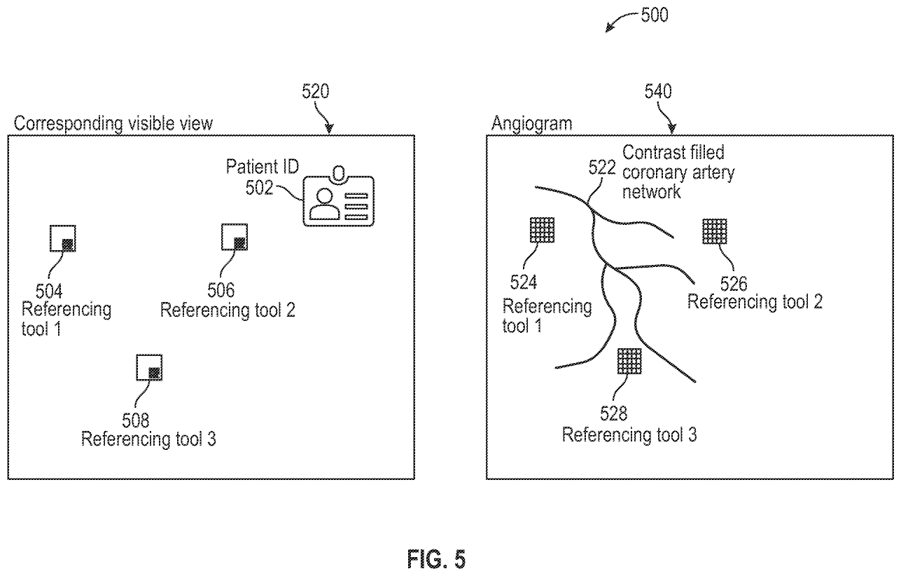

FIG. 5 shows a set of representations of exemplary diagrams 500 of multiple reference objects that can be used by the disclosed exemplary apparatus/system/method/device/computer-accessible medium in a visible field of view according to yet another exemplary embodiment of the present disclosure. A diagram 520 is an illustration of an angiogram that can comprise, e.g., in addition to a patient ID 502, a plurality of reference objects/tools, such as, e.g., a first reference object 504, a second reference object 506, and a third reference object 508. The patient ID 502 can contain or be associated with information on a patient such as name, age, and so forth. Each of the reference objects 504, 506, 508 can further comprise a scannable ID, such as, e.g., a QR code which can be associated with the information regarding the imaging system, the patient and/or the procedure. A diagram 540 is an illustration of an angiogram that shows, in addition to a contrast filled coronary artery network 522, a plurality of reference objects/tools such as a first reference object 524, a second reference object 526, and a third reference object 528. Each of the reference objects 524, 526, 528 can be arranged as a grid, and the plurality of reference objects can be arranged in any suable position.

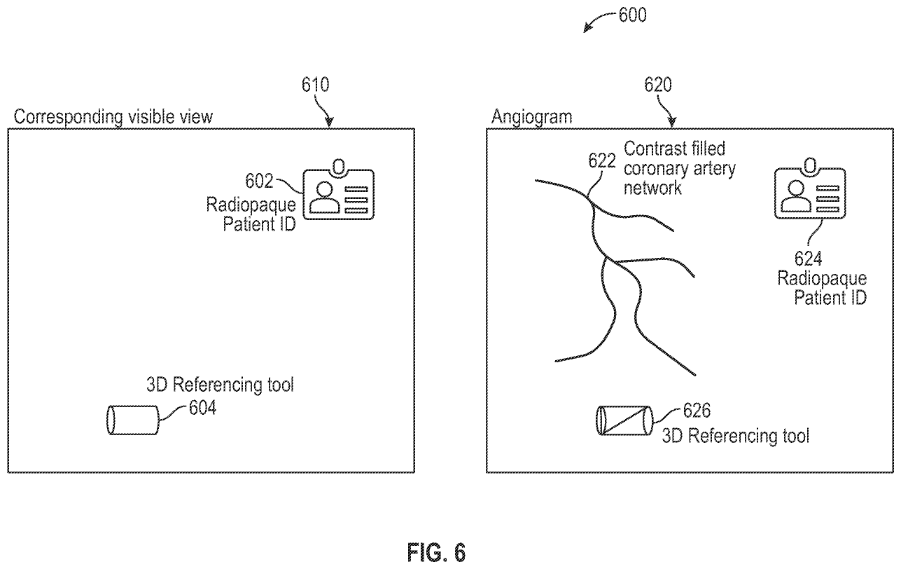

In some exemplary embodiments of the present disclosure, a reference object can be a 3D object. FIG. 6 shows as set of exemplary illustration of exemplary diagrams 600 providing 3D reference objects that can be used by the exemplary apparatus/system/method/device/computer-accessible medium in a visible field of view according to a further exemplary embodiment of the present disclosure. A diagram 610 is an illustration of an angiogram that can comprise a patient ID 602, and a 3D reference object/tool 604. The patient ID 602 can contain or be associated with information on a patient such as name, age, and so forth. The 3D reference object 604 can further comprise a scannable ID such as a QR code in which information on the imaging system can be stored. A diagram 620 is an illustration of an angiogram that shows a 3D reference object 626 in addition to a contrast filled coronary artery network 622 and a radiopaque patient ID 624.

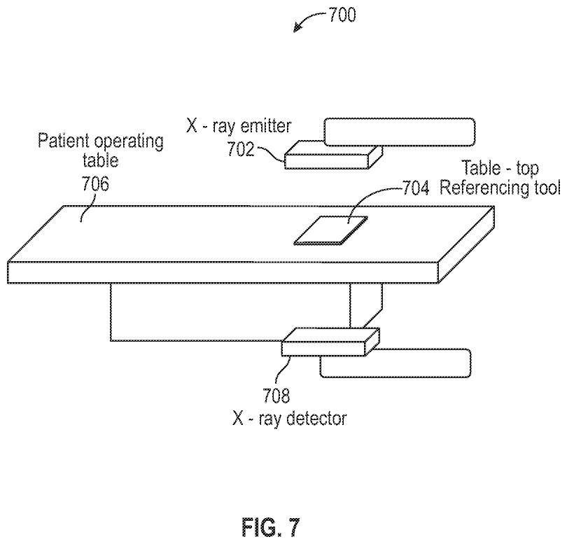

FIG. 7 illustrates a diagram of an exemplary apparatus/system 700 which has or utilizes a reference object for imaging patients according to another exemplary embodiment of the present disclosure. The exemplary system 700 can comprise an X-ray emitter 702 that can be configured to image at least one portion of a patient, a reference object 704 placed on top of a platform 706 (e.g., an operating table) on which the patient can be placed in a, e.g., horizontal position, and an X-ray detector 708 that can be configured to detect X-ray transmitted through the platform 706, the portion(s) of the patient and the reference object 704 to obtain the information regarding the patient and the reference object so as to facilitate a formation an X-ray image of an associated processor.

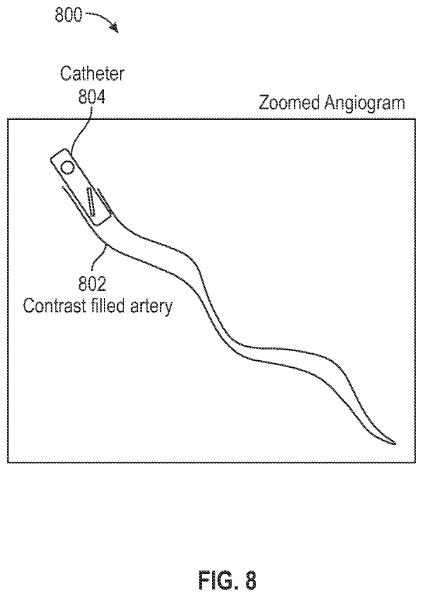

FIG. 8 shows a diagram 800 of an exemplary angiogram showing an intravascular device, in this exemplary case-a catheter 804, and a progression thereof within a lumen of a body that can be implemented in the disclosed apparatus/system/computer-accessible medium according to yet another exemplary embodiment of the present disclosure. A pattern disposed on the catheter 804 (e.g., diagnostic catheter, guide catheter, imaging catheter)-which can be known or unknown—may be used to obtain pixel dimensions in order to do real-time ADCA, e.g., during a diagnostic angiogram procedure, during a PCI procedure, etc., Similarly, one or more dimensions of the catheter itself (e.g., without a pattern) can be used to calibrate an image, e.g., to obtain pixel dimensions, for ADCA. The exemplary diagram 800 also provides a contrasted filled artery 802 in which the catheter 804 can be positioned. In some exemplary embodiments of the present disclosure, a user can input or otherwise provide dimension(s) of the catheter (e.g., the inner diameter, the outer diameter, length, etc.) to aid a calibration procedure. In addition or alternatively, the user input can include inputting any and/or combination of:
catheter manufacturer, model number or identification, etc.
the type of catheter (e.g. a guide catheter, a diagnostic catheter, an imaging catheter, etc.)
a unit of measure for the guide catheter (e.g., an OD of: 3Fr, 4 Fr, 5Fr, 6Fr, 7Fr, 8Fr, 9Fr, 10Fr)

According to various exemplary embodiments of the present disclosure, the intravascular device can be automatically detected and/or segmented (e.g. using standard image processing techniques, machine learning procedures, deep learning procedures, neural network(s) such as a U-net, ResNet, etc.) in order to determine and/or calculate the total number of pixels along an axis, e.g., with the perpendicular axis to the devices longitudinal direction being used to measure the diameter. In another exemplary embodiment of the present disclosure, a user can input or otherwise provide the size of the intravascular object, and manually or automatically annotate a dimension of the reference object to extract pixel sizes and/or calibrate an image. In still another exemplary embodiment of the present disclosure, a physical offset (e.g., specific to the object, the x-ray system, the patient) can be applied to the user-inputted size, such as e.g., outer diameter, inner diameter, length, etc. of a guide catheter, to account for difference between visible and invisible portions of the object on an x-ray image such as e.g., user-inputted outer diameter of a diagnostic/guide catheter and the visible portion of a diagnostic/guide catheter in an x-ray image and/or the contrast-filled lumen. According to further exemplary embodiments of the present disclosure, a processor, as described herein, can be connected to a storage device containing information about the x-ray system from which the x-ray data originates (e.g., stored from a previous time point, provided during an install/configuration timepoint, etc.) and this information can also be used to assist in calibrating the x-ray data, e.g., along with a user input and an automatically detected reference object.

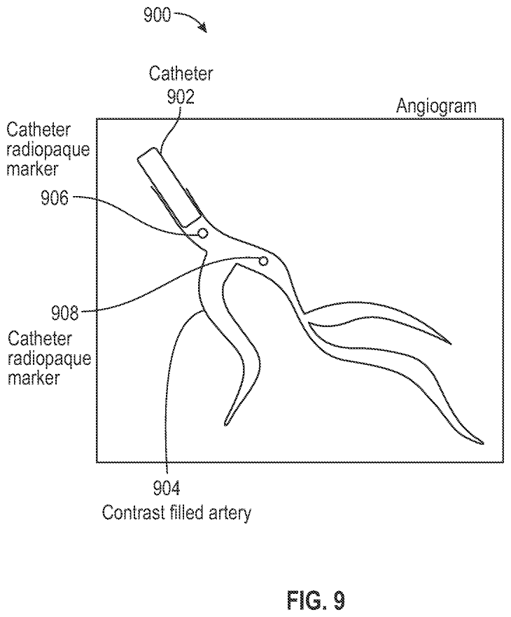

In addition to a catheter (e.g., a guide catheter) that can be used as a reference object, in some exemplary embodiments of the present disclosure, one or more additional reference objects can be used for determining image pixel size. For example, FIG. 9 shows a diagram 900 of an exemplary angiogram showing markers that can be implemented in the apparatus/system/computer-accessible medium according to yet another exemplary embodiment of the present disclosure. As shown in FIG. 9, the diagram 900 includes two catheter radiopaque markers 906, 908 which can be used as reference objects for obtaining image pixel sizes, in addition to a contrast-filled artery 904 in which the guide catheter 902 and the two catheter radiopaque markers 906, 908 can be positioned. The catheter radiopaque markers 906, 908 can be placed in the field of view with a known size or a known pattern to obtain pixel dimension(s) in order to perform real-time imaging. In this example, the catheter radiopaque markers 906, 908 can be placed on or in another catheter (e.g., an imaging catheter) which may not be radiopaque, such that such other catheter may not be visible except for the catheter radiopaque markers 906, 908.

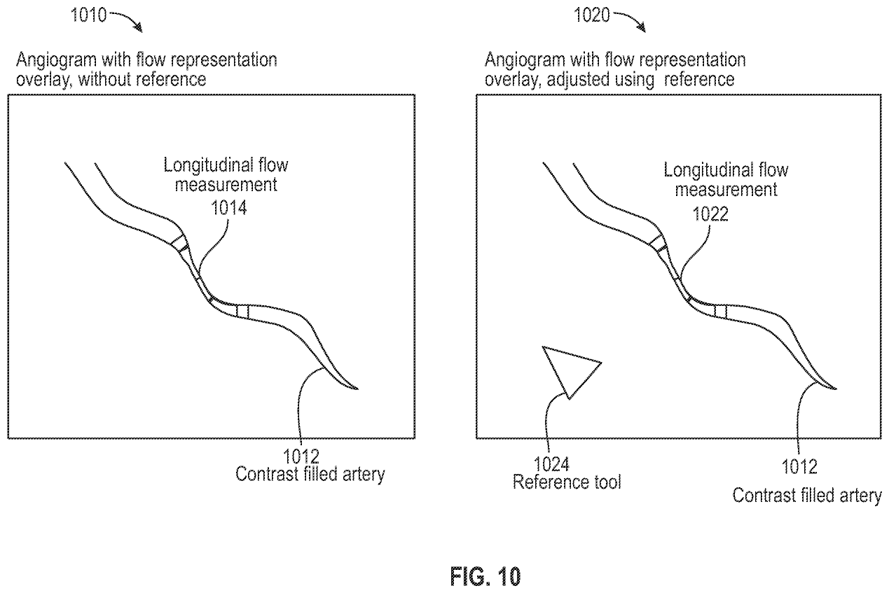

FIG. 10 shows a set illustration of exemplary angiograms providing exemplary ADP measurement results with and without a reference object that can be implemented in the system/apparatus/device/method/computer-accessible medium according to an exemplary embodiment of the present disclosure. In particular, the exemplary angiogram diagram 1010 of FIG. 10 is obtained without a reference object, and shows a first longitudinal ADP measurement 1014 overlaying a contrast filled artery 1012 without a reference object. The angiogram diagram 1020 is obtained with a reference object, and shows a second longitudinal ADP measurement 1022 overlaying the contrast filled artery 1012 with a reference object 1024. The second longitudinal ADP measurement 1022 can be determined based on the radiation obtained in conjunction with the reference object 1024, and as shown in FIG. 10, can be more accurate than the first longitudinal ADP measurement 1014.

According to exemplary embodiments of the present disclosure, ADP parameters can be derived through the automatic detection of geometric or dynamic (e.g. flow) information from contrast filled arteries. ADP parameters can be based on computational fluid dynamics (e.g. 3-dimension, 2-dimensional, 1-dimensional, 0-dimensional, etc.). ADP parameters can also be based any closed-form, deterministic formulations of the physical laws of fluid dynamics, governing the incompressibility of fluids in a closed system (e.g., Navier-stokes, Bernoulli and Poiseuille). Moreover, ADP parameters can be empirical or data-driven, based on measurements of geometric or dynamic properties and compared (e.g., trained) against ground truth measurements (e.g., using a pressure wire).

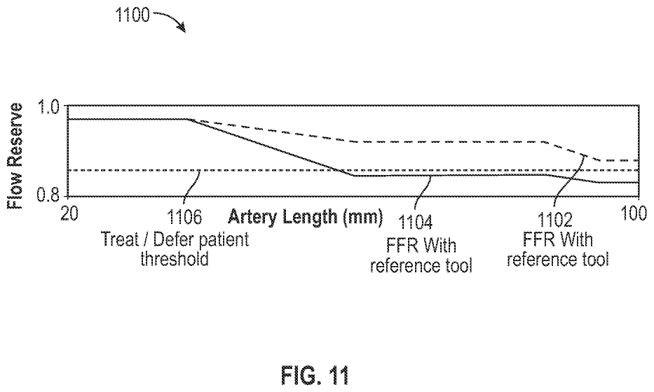

FIG. 11 shows a cross-sectional view of an illustration/diagram 1100 providing further ADP measurement results with and without a reference object, respectively, that is implemented in the system/apparatus/device/method/computer-accessible medium according to an exemplary embodiment of the present disclosure. The exemplary diagram 1100 illustrates an ADP reserve measurement 1102 without a reference object, as well as an ADP reserve measurement 1104 with a reference object. The diagram 1100 also shows a treat/defer patient threshold 1106. As can be seen in FIG. 11, the ADP reserve measurement 1102 without a reference object is greater than the treat/defer patient threshold 1106, whereas the ADP reserve measurement 1104 with a reference object is smaller than the treat/defer patient threshold 1106. Therefore, the presence of the reference object can impact (e.g., improve) the treatment of the patient by providing more accurate measurements.

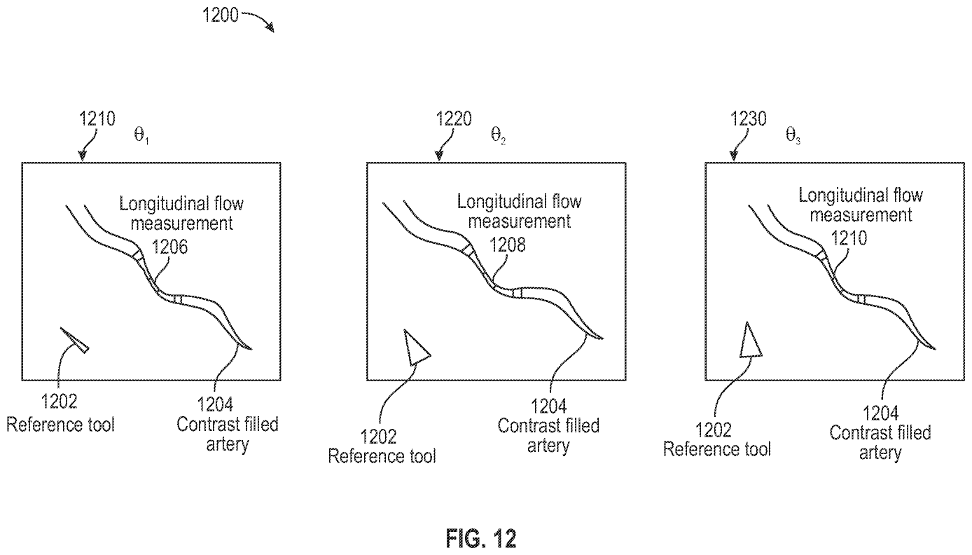

FIG. 12 shows a set of diagrams 1200 (including diagrams 1201, 1220, 1230) providing exemplary ADP measurements with different placement angles of a reference object that is implemented in the system/apparatus/device/method/computer-accessible medium according to an exemplary embodiment of the present disclosure. The diagram 1210 is, e.g., a diagram of a longitudinal ADP measurement 1206 in a contrast-filled artery 1204 with a reference object 2102 having a placement angle of θ₁. The diagram 1220 is, e.g., a diagram of a longitudinal flow measurement 1208 in the contrast filled artery 1204 with the reference object 2102 having a placement angle of θ₂. The diagram 1220 is a diagram of a longitudinal ADP measurement 1210 in the contrast-filled artery 1204 with the reference object 2102 having a placement angle of θ₃. As can be seen in FIG. 12, the placement of the reference object is invariant to angle, and therefore facilitate a compensation for an angular variation in the imaging parameters, e.g., for consistent longitudinal ADP measurements.

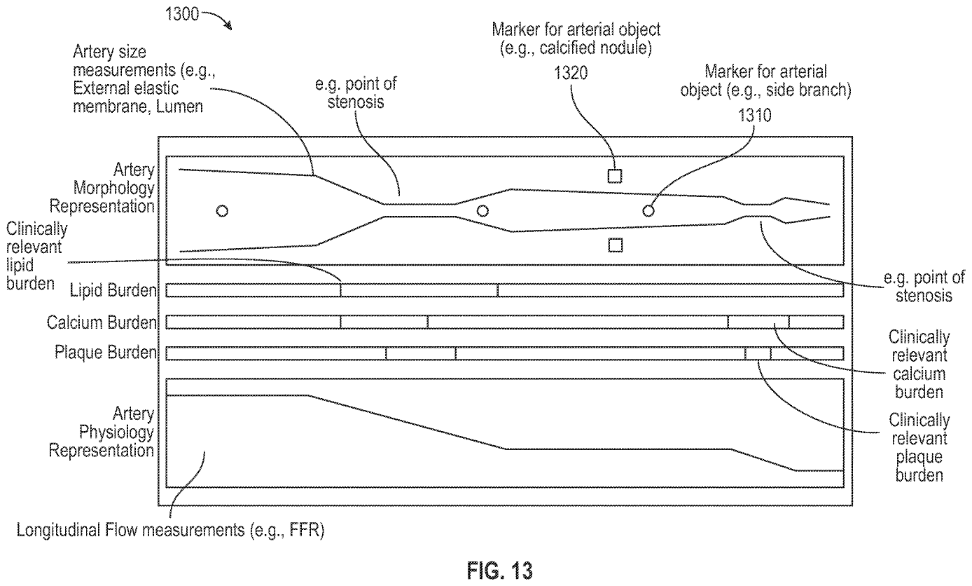

FIG. 13 shows a cross-sectional view of a set of illustrations/diagrams 1300 providing still further reference objects used for multiple imaging modes that are implemented in the system/apparatus/device/method/computer-accessible medium according to an exemplary embodiment of the present disclosure. As shown in FIG. 13, exemplary reference objects (i.e., markers) 1310 can be replaced inside an artery for side branch detection(s), and reference objects (i.e., markers) 1320 can be replaced outside an artery for calcified nodule detection(s).

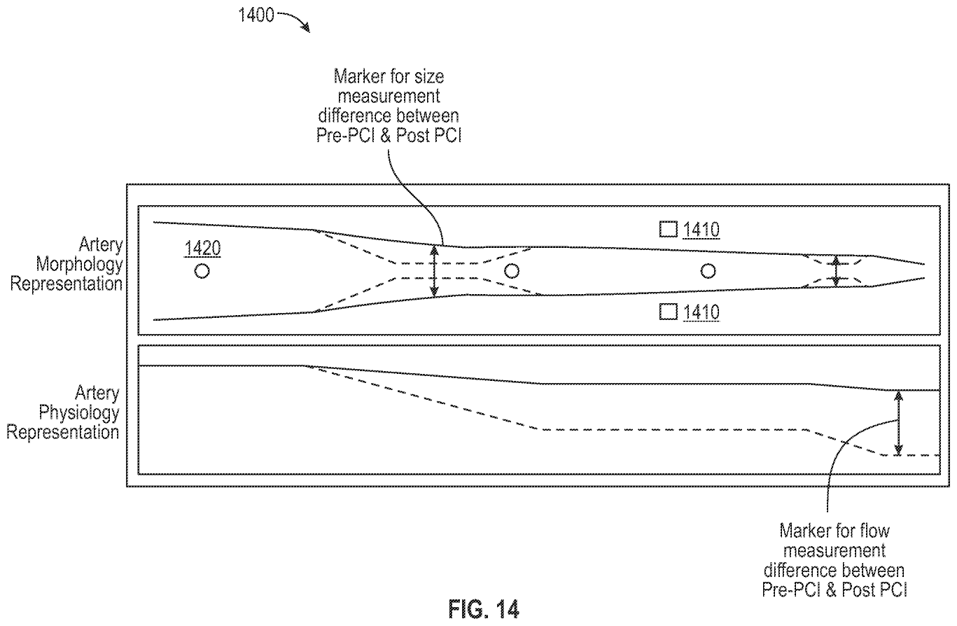

FIG. 14 shows a cross-sectional view of a set of illustrations/diagrams 1400 providing yet further reference objects used for multiple imaging modes that are implemented in the system/apparatus/device/method/computer-accessible medium according to another exemplary embodiment of the present disclosure. As shown in FIG. 14, exemplary reference objects (e.g., markers) 1410, 1420 can be used for determining a size measurement difference between pre-percutaneous coronary intervention (PCI) and post PCI, and/or for ADP measurement difference between pre-PCI and post PCI.

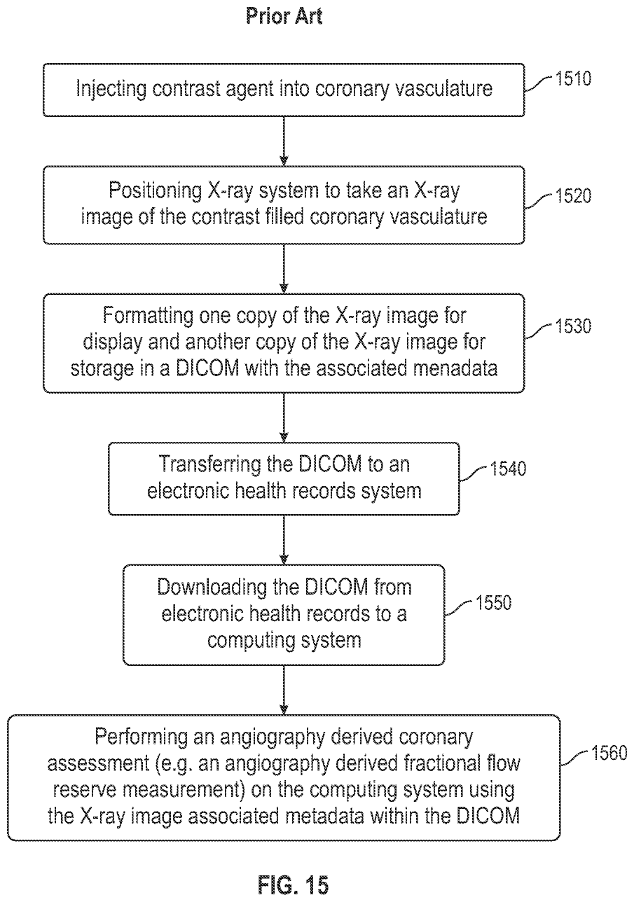

FIG. 15 shows a flow diagram for computing a fractional flow reserve (FFR) based on the display image as exists in the prior art. In particular, according to this prior art method, contrast agent is injected into coronary vasculature (procedure 1510). The X-ray system is the positioned to take an X-ray image of the contrast-filled coronary vasculature (procedure 1520). Then, one copy of the X-ray image is formatted for display, and another copy of the X-ray image is formatted for storage in a DICOM with the associated meta data (procedure 1530). The DICOM is then transferred to an electronic health records system (EHS) (procedure 1540), and the such DICOM file is downloaded to a computing system (procedure 1550). Finally, an angiography derived coronary assessment (e.g., an angiography derived fractional flow reserve measurement) can be performed on the computing system using the X-ray image associated metadata within the DICOM (procedure 1560). Thus, this prior art method would cause the data to be transmitted from the X-ray system to the HER system, and then back to the computing system, which collectively would incur high data-message delay (e.g., high-latency).

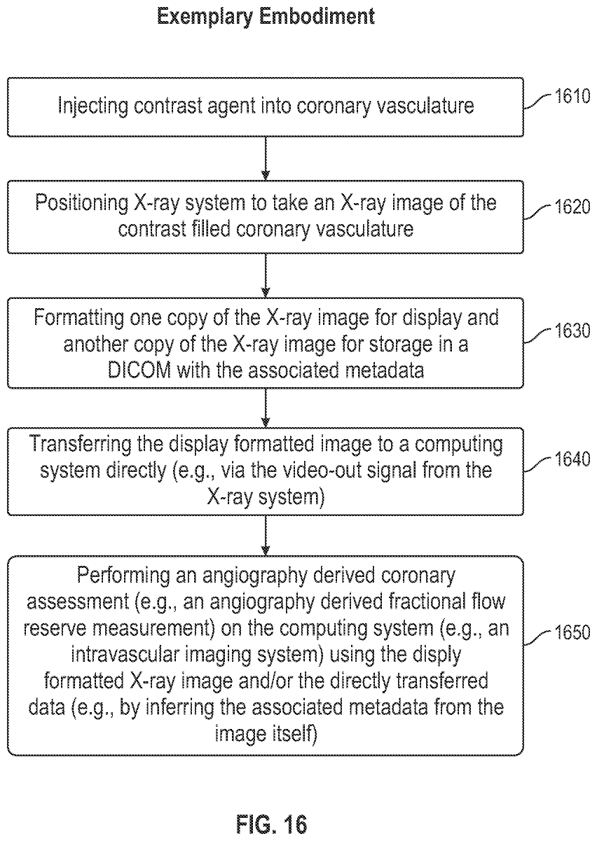

To address this issue, an exemplary process according to exemplary embodiment of the present disclosure can be provide, as shown in a flow diagram of FIG. 16. This exemplary process computes the fractional flow reserve (FFR) based on the display image from the video out port on an intravascular imaging system that are implemented in the system/apparatus/device/method/computer-accessible medium according to another exemplary embodiment of the present disclosure. In particular, similarly to the prior art process shown in FIG. 15, in exemplary process of FIG. 16, contrast agent is injected into coronary vasculature (procedure 1610), the X-ray system is the positioned to take an X-ray image of the contrast-filled coronary vasculature (procedure 1620), and one copy of the X-ray image is formatted for display, and another copy of the X-ray image is formatted for storage in a DICOM with the associated meta data (procedure 1630). Then, in contrast to the prior art process of FIG. 15, the exemplary process transfers the display formatted image to computing system directly (procedure 1640), e.g., as video-out data. Finally, the angiography derived coronary assessment (e.g., the angiography derived fractional flow reserve measurement) can be performed on the computing system (e.g., an intravascular imaging system) using the display formatted X-ray image and/or directly-transferred data (e.g., by inferring the associated metadata from the image itself) (procedure 1650). In summary, the exemplary process shown in FIG. 16 can utilize a “frame-grabber” to, e.g., stream video-out data from the X-ray system to the computing system, and process this display-formatted image. For example, the transfer can be low latency (e.g., less than 1 second/data-message). In certain exemplary embodiments of the present disclosure, the angiography derived coronary assessment can be performed with image data from a single X-ray angle. According to further exemplary embodiments of the present disclosure, the angiography derived coronary assessment can be performed with image data from a sequence of images taken at more than one X-ray angle.

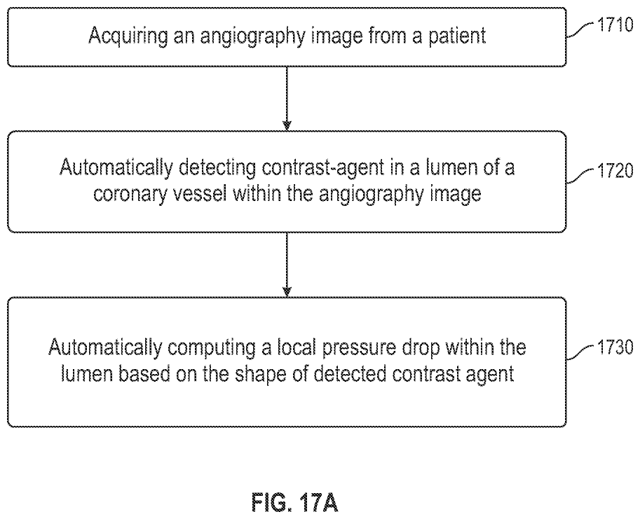

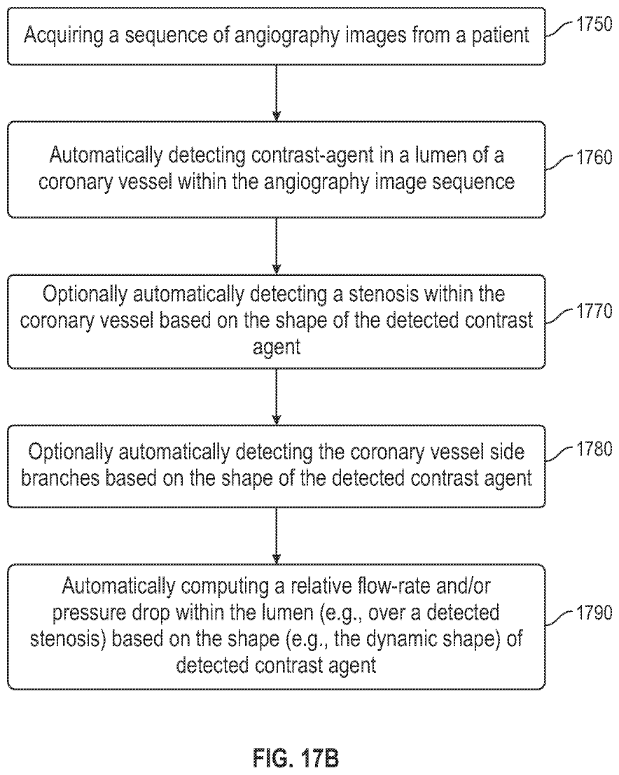

FIG. 17A shows a flow diagram of a process for obtaining and analyzing angiography-derived measurements based on contrast agent filling lumen of a vessel according to an exemplary embodiment of the present disclosure. In particular, the angiography image is acquired from the patient, e.g., using the X-ray system (procedure 1710). Then, the contrast-agent in a lumen of a coronary vessel within the angiography image is automatically detected (procedure 1720). Finally, in this exemplary process, a local pressure drop within the lumen is automatically determined and/or computer based on the shape of detected contrast agent (procedure 1730).

FIG. 17B shows a flow diagram of a method that utilizes at least one image sequence to measure flow and pressure, optionally incorporating side branches which can also cause a pressure drop that can improve the accuracy of the FFR measurement according to an exemplary embodiment of the present disclosure. For this exemplary process, the angiography image is acquired from the patient, e.g., using the X-ray system (procedure 1750). The contrast-agent in a lumen of a coronary vessel within the angiography image segments is automatically detected (procedure 1760). Further, a stenosis within the coronary vessel is automatically detected based on the shape of the detected contrast agent (procedure 1770). Additionally, the coronary vessel side branches is automatically detected based on the shape of the detected contrast agent (procedure 1780). Finally, in this exemplary process, a relative flow-rate and/or pressure drop within the lumen (e.g., over a detected stenosis) can be automatically computed or determined based on the shape (e.g., the dynamic shape) of detected contrast agent (procedure 1790).

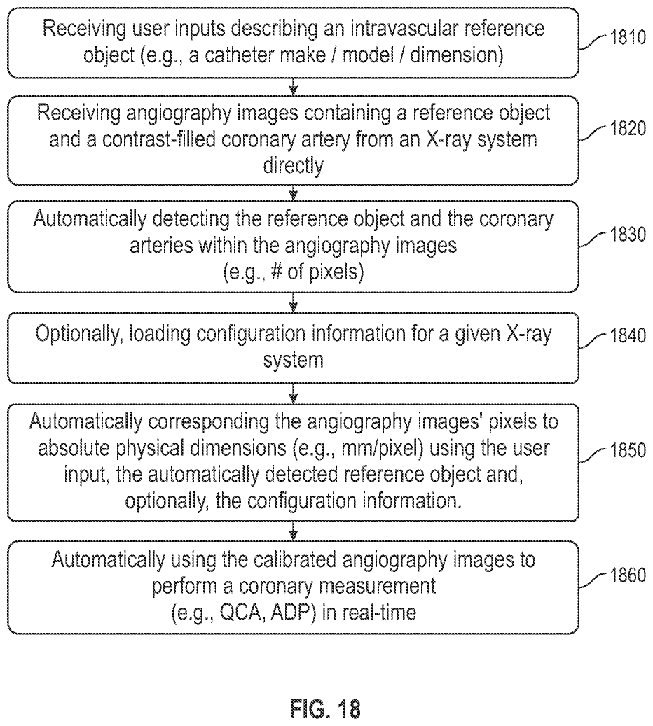

FIG. 18 shows a flow diagram of a method according to an exemplary embodiment of the present disclosure that utilizes at least one angiography image to perform an accurate angiography measurement (e.g., QCA, or ADP). As shown in FIG. 18, a user-input can be incorporated and/or utilized (procedure 1810). For this exemplary process, the angiography image can acquired from the patient, e.g., using the X-ray system (procedure 1820). The lumen of a coronary vessel (e.g., filled with contrast agent) within the angiography image segments can be automatically detected, along with a reference object, such as e.g., a diagnostic catheter) (procedure 1830). Optionally, X-ray system information can be received from a storage device that can provide information about the connected X-ray system, such as e.g., information on the X-ray detector's pixel sizes (procedure 1840). Further, in this exemplary process, an automatically computed calibration procedure can be performed (procedure 1850) in order to perform accurate coronary measurements (procedure 1860) (e.g., in real-time, e.g., during a PCI procedure).

As described as exemplary embodiments herein, an imaging system/apparatus/device and an imaging method implemented thereon can comprise a X-ray radiation source, one or more reference objects (such as, tools, markers) deployed in the imaging view field of the X-ray radiation source, and a X-ray detector or imaging device. The one or more reference objects are radiopaque reference objects. The one or more reference objects can be placed in any suitable position as long as they are in the imaging view field of the X-ray imaging system, such as on a table in an operating room, on an imaging catheter, and so forth. Image pixel sizes and information of the X-ray imaging system can be obtained from the one or more reference objects, such as a distance between the X-ray radiation source and a patient, a distance between the X-Ray detector/imaging device and the patient, and so forth. For example, a radiopaque contrast agent can be deployed in a body, e.g., in an imaging view field of the X-ray radiation source, so that the at least one portion of the body can be easier detected using X-ray radiation provided from such portion and the radiation associated with the reference objects,

As described as additional exemplary embodiments herein, a method for tracking software use in an imaging system can be provided based on a reference object. The reference object can be provided in or on at least one section of a body or an object which can be used to generate an X-ray image of at least one portion of the body or the object. Additional information can be retrieved from the reference object being imaged to identify and/or track, for example, information associated with the object or the body (e.g., patient data), as well as, e.g., payment/charge for using the software by the patient. The reference object can be a marker, a symbol or any combinations, such as a QR code.

Exemplary embodiments of the present disclosure provide an imaging system which can comprises, e.g., a X-ray radiation source, at least one radiopaque reference object provided within an imaging field-of-view of the X-ray radiation source, and an X-ray detector which detects the X-ray radiation exiting at least one portion of a body or an object, and generates associated information, and at least one computer processor configured to generate a X-ray image containing an image of the at least one portion and the radiopaque reference object based on the associated information.

As used in the present disclosure, a computer can include any device (which can have a processor) that can be configured to receive information, and, e.g., effectuate the generation or the output of the electromagnetic radiation if the rate of motion is greater than a predetermined rate based on the information. The computer can also include storage device(s) (e.g., memory, hard drive, RAM, ROM, removable storage devices, etc.), as well as network connectivity ports for receiving and transmitting data. The computer can also be or include a microprocessor, a logic circuit, etc.

Throughout the disclosure, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “some examples,” “other examples,” “one example,” “an example,” “various examples,” “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described may include a particular feature, structure, or characteristic, but not every implementation necessarily includes the particular feature, structure, or characteristic. Further, repeated use of the phrases “in one example,” “in one exemplary embodiment,” or “in one implementation” does not necessarily refer to the same example, exemplary embodiment, or implementation, although it may.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the appended claims, and may include other examples that occur to those skilled in the art. Such other examples are intended to be within the scope of the appended claims if they have structural elements that do not differ from the literal language of the appended claims, or if they include equivalent structural elements with insubstantial differences from the literal language of the appended claims.

What is claimed is:

1. An imaging system, comprising:
at least one partially radiopaque reference object provided within an imaging field-of-view of an X-ray radiation source and in vicinity of at least one portion of a body or a target object, and configured to receive an X-ray radiation generated by the X-ray radiation source; and
at least one computer processor configured to generate an X-ray image of vasculature of the at least one portion of the body containing an image of the reference object based on (i) a first radiation provided from the at least one portion, and (ii) a second radiation provided from the reference object.

2. The system of claim 1, wherein the at least one computer processor is configured to generate an X-ray image after a partially radiopaque contrast agent is provided into the at least one portion.

3. The system of claim 1, further comprising an X-ray detector in at least one of optical communication or data communication with the at least one computer processor.

4. The system of claim 1, wherein the at least one reference object is a 2-dimensional object or a three-dimensional object.

5. The system of claim 4, wherein the at least one reference object is at least one of a triangle-shaped object, a square-shaped object, a grid-shaped object, or a polygonal-shaped object.

6. The system of claim 1, wherein the at least one reference object is arranged as a pattern.

7. The system of claim 1, wherein the at least one reference object is provided on a platform on which the at least one portion is positioned.

8. The system of claim 1, wherein the at least one reference object is provided within the portion of the body.

9. The system of claim 1, wherein the at least one reference object is provided on a an imaging catheter.

10. The system of claim 1, wherein the at least one reference object is provided on a guide catheter.

11. The system of claim 1, wherein the at least one reference object is provided on at least one of a front section or a back portion of the body or the target object.

12. The system of claim 1, wherein the at least one reference object comprises an image-readable identification code.

13. The system of claim 12, wherein the image-readable identification code has 3-dimensional characteristics.

14. The system of claim 12, wherein the 3-dimensional characteristics are usable to encode at least one portion of the information in an attenuation of X-ray through each of the 3-dimensional characteristics or one or more locations thereof.

15. The system of claim 1, wherein the at least one reference object is at least partially composed of a metal material and or a polymer material.

16. The system of claim 1, wherein the at least one computer processor is configured to generate to obtain patient metadata from the image of the radiopaque reference object.

17. The system of claim 1, wherein pixel sizes of the X-ray image are obtained from the image of the radiopaque reference object.

18. The system of claim 1, wherein a second computer processor is configured to automatically detect the reference object within the X-ray image.

19. The system of claim 1, wherein a second compute processor is configured to automatically detect a contrast-filled coronary artery from the first portion within the X-ray image.

20. The system of claim 19, wherein the computer processor is configured to measure physiologic information based on information from the detected reference object and the detected contrast-filled coronary artery.

21. The system of claim 20, wherein physiologic information may include a measure of flow rate, factional flow reserve, coronary flow reserve or an index of microcirculatory resistance.

22. The system of claim 1, wherein at least one computer processor is configured to extract information from the at least one X-ray image.

23. The system of claim 22, wherein the extracted information is provided from the reference object and the vasculature.

24. The system of claim 22, wherein the at least one computer processor is further configured to measure a physiological index or a structural index based on the extracted information.

25. The system of claim 24, wherein the physiological index includes at least one of a flow rate, a fractional flow reserve, a coronary flow reserve or an index of microcirculatory resistance.

26. The system of claim 22, wherein the structural index includes at least one of a stenosis, an apposition, a distance, a size or a length of at least one section of a vessel.

27. The system of claim 1, further comprising a user interface which is configured to facilitate a user input to provide user data to the at least one computer processor.

28. The system of claim 27, wherein the user data includes at least one of anatomic information, physiologic information or reference object information.

29. The system of claim 28, wherein the at least one computer processor is further configured to automatically measure at least one geometric data or physiology data for at least one artery of the vasculature based on the user data.

30. The system of claim 20, wherein the at least one computer processor is configured to determine physiologic information using a computational fluid dynamics assessment of at least one artery of the vasculature.

31. The system of claim 20, wherein the at least one computer processor is configured to determine physiologic information using a machine-learned assessment of at least one artery of the vasculature.

32. A method of performing image-based coronary assessments within a body, comprising:
disposing at least one reference object within an imaging field-of-view of an X-ray radiation generator, and in vicinity of at least one portion of a body or a target object;
providing an X-ray radiation to at least one portion of a body or a target and the at least one reference object; and
generating at least one X-ray image of the at least one portion containing an image of the reference object based on (i) a first radiation provided from the at least one portion, and (ii) a second radiation provided from the reference object.

33. The method of claim 32, further comprising providing a contrast agent into or near the at least one portion while generating the at least one X-ray image.

34. The method of claim 32, further comprising measuring a physiological feature of the at least one portion based on information from both the first radiation and the second radiation.

35. The method of claim 32, wherein a physiological feature may include an index of flow rate, fractional flow reserve, coronary flow reserve or an index of microcirculatory resistance.

36. The method of claim 32, wherein the at least one reference object is a patterned object having known properties.

37. A method for tracking use of a technology, the method comprising:
disposing a partially radiopaque reference object within an imaging field-of-view of an X-ray light generator, and in vicinity of at least one portion of a body or a target object; and
facilitating image-based detection and tracking of the target object using a first X-ray radiation received from the at least one portion, and a second X-ray radiation received from at least one interpretable symbol of the reference object.

38. The method of claim 37, further comprising obtaining financial information based on the tracking of the body or the target object.

39. A method of performing real-time image-based coronary assessment, comprising:
providing an X-ray radiation to at least one portion of a body
generating at least one X-ray image of the at least one portion after a partially radiopaque contrast agent is provided in or on the at least one portion;
transmitting the X-ray image directly to a processor;
measuring a physiology index based on the X-ray image; and displaying a representation of the physiology index on a display.

40. The method of claim 37, wherein the X-ray image is transmitted via a wired connection or a wireless connection from a data streaming port (e.g., which can be a video out port) of an X-ray apparatus.

41. The method of claim 37, wherein the X-ray image is not a DICOM standard image.

42. The method of claim 37, further comprising utilizing sequential images (e.g., from more than one X-ray angle) to improve or adjust the measurement.

43. The method of claim 37, further comprising performing the image-based coronary assessment at a single X-ray imaging angle.

44. The method of claim 37, further comprising utilizing a reference object to adjust the measurement.

45. A real-time image-based coronary assessment system, comprising:
an X-ray apparatus configured to provide an X-ray radiation to at least one portion of a body, and generate at least one X-ray image of the at least one portion after at least partially radiopaque contrast agent is provided into the at least one portion; and
a computer processor configured to receive the X-ray image directly from the X-ray apparatus and extract information from the directly-obtained X-ray image.

46. The system of claim 45, wherein the information includes a physiology or structural measurement of the at least one portion.

47. The system of claim 46, further comprising a display configured to provide a first representation of the at least one portion within the X-ray image and a second representation of the physiology or structural measurement.

48. The system of claim 45, wherein the X-ray image is received via a video out signal.

49. A method for providing a real-time image-based coronary assessment, comprising:
providing an X-ray radiation to at least one portion of a body by an X-ray apparatus;
generating at least one X-ray image of the at least one portion after at least partially radiopaque contrast agent is provided into the at least one portion; and
with a computer processor, (i) receiving the X-ray image directly from the X-ray apparatus, and (ii) extracting information from the directly-obtained X-ray image.

50. The method of claim 49, wherein the information includes a physiology or structural measurement of the at least one portion.

51. The method of claim 50, further comprising a display configured to provide a first representation of the at least one portion within the X-ray image and a second representation of the physiology or structural measurement.

52. The method of claim 49, wherein the X-ray image is received via a video out signal.

53. A method of performing angiography image referencing, the method comprising:
providing a partially radiopaque contrast agent within a bodily lumen;
disposing a partially radiopaque reference object in the vicinity of the body lumen, imaging a portion of the bodily lumen and the radiopaque reference object, detecting a portion of the contrast agent and the reference object automatically within the image.

54. The method of claim 53, further comprising displaying the image.

55. The method of claim 53, further comprising adjusting the display of the image based on the automatically detected contrast agent and reference object.

56. The method of claim 53, further comprising measuring a physiological feature of the bodily lumen based on the automatically detected contrast agent and reference object.

57. A method for tracking use of a technology, the method comprising:
disposing a partially radiopaque reference object within an imaging field-of-view wherein,
the radiopaque reference object comprises an image-readable symbol or combination of symbols that allows tracking of its use.

58. The method of claim 57, further comprising charging a payment based on the tracking.

59. A measurement system, comprising:
at least one partially radiopaque reference object provided within an imaging field-of-view of an X-ray radiation source and in vicinity of at least one portion of a body or a target object, and configured to receive the X-ray radiation;
a data interface; and
at least one computer processor configured to:
receive, directly via the data interface, at least one X-ray image of vasculature of the at least one portion of the body containing at least part of an image of the reference object;
calibrate the dimensions of the at least one X-ray image based on the reference object.

60. The system of claim 59, wherein the at least one computer processor is configured to automatically detect the reference object in the image.

61. The system of claim 59, wherein the at least one computer processor is configured to automatically detect the vasculature in the image.

62. The system of claim 59, wherein the reference object is an intravascular device.

63. The system of claim 59, wherein the reference object is a catheter.

64. The system of claim 59, wherein the reference object is an extravascular device.

65. The system of claim 59, wherein the at least one computer processor is part of an measurement system which is an intravascular imaging system.

66. The system of claim 59, further comprising a user interface configured to receive user-inputted information.

67. The system of claim 66, wherein the user-inputted information which is at least one of anatomic information, physiologic information, or reference object information.

68. The system of claim 66, wherein the user-inputted information is usable, at least in part, to calibrate the X-ray image.

69. The system of claim 59, wherein at least one computer processor is configured to extract information from the at least one X-ray image.

70. The system of claim 59, wherein the extracted information is provided from the reference object and the vasculature.

71. The system of claim 70, wherein the at least one computer processor is further configured to provide an image-based coronary assessment based on the extracted information.

72. The system of claim 71, wherein the image-based coronary assessment is a physiological measurement or a structural measurement.

73. The system of claim 72, wherein the physiological measurement includes at least one of a flow rate, a fractional flow reserve, a coronary flow reserve or an index of microcirculatory resistance.

74. The system of claim 72, wherein the structural measurement includes at least one of a stenosis, a position, a distance, a size or a length of at least one section of a vessel.

75. The system of claim 59, wherein the at least one X-ray image excludes a DICOM standard image.

76. The system of claim 71, wherein the at least one computer processor is further configured to control or adjust the assessment by utilizing sequential images from more than one of the at least one X-ray image.

77. The system of claim 71, wherein the at least one computer processor is further configured to perform the image-based coronary assessment at a single X-ray imaging angle.

78. The system of claim 71, wherein the at least one computer processor is further configured to adjust the assessment using a reference object.

79. The system of claim 59, wherein the data interface is a data transfer cable.

80. An imaging system, comprising:
at least one insertion device structured to be provided into a body and including at least one section which is configured to receive the X-ray radiation; and
at least one computer processor configured to generate an X-ray image of vasculature of the at least one portion of the body containing an image of at least one portion of the body based on (i) a second radiation provided from the at least one section; and (ii) information regarding the at least one insertion device.

81. The system of claim 80, wherein the at least one computer processor is further configured to determine information regarding at least one coronary measurement from the X-ray image of the vasculature.

82. The system of claim 81, wherein the at least one computer processor determines the information regarding the at least one coronary measurement by automatically detecting at least one geometric data or dynamic data for at least one artery of the vasculature.

83. The system of claim 82, wherein the information includes one or more physiological parameters of the at least one artery.

84. The system of claim 80, wherein the at least one computer processor is further configured to determine information regarding physiology from the X-ray image of the vasculature.

85. The system of claim 80, wherein the at least one computer processor is configured to determine the information regarding the physiology based on computational fluid dynamics data from at least one artery of the vasculature.
